(19) United States
(12) Patent Application Publication
(10) Pub. No.: US 2022/0304353 A1
(43) Pub. Date: Sep. 29, 2022

(54) Electrolyte Beverage Composition

(71) Applicant: Health Well CBD Corp., Fort Lauderdale, FL (US)

(72) Inventor: Steven Adelstein, Fort Lauderdale, FL (US)

(73) Assignee: Health Well CBD Corp., Fort Lauderdale, FL (US)

(21) Appl. No.: 17/707,687

(22) Filed: Mar. 29, 2022

Related U.S. Application Data
(60) Provisional application No. 63/167,478, filed on Mar. 29, 2021.

(51) Int. Cl. A23L 29/00; A23L 33/105; A23L 33/10; A23L 2/52; A23L 2/395; A23L 33/15; A23L 33/16; A61K 31/05; A61K 31/375; A61K 33/00; A61K 36/77

(52) U.S. Cl. CPC A23L 29/035; A23L 33/105; A23L 33/10; A23L 2/52; A23L 2/395; A23L 33/15; A23L 33/16; A61K 31/05; A61K 31/375; A61K 33/00; A61K 36/77

(57) ABSTRACT

An ingestible composition and method for providing electrolytes and for other beneficial health effects related to digestion, nutrient absorption, pain prevention and alleviation, blood pressure reduction, and anxiety relief. The composition includes minerals, mastica, vitamin C, and a cannabinoid compound. The cannabinoid compound can be cannabidiol or another cannabinoid. The composition can be produced in solid form such as a powder or in liquid form as a beverage.

### CROSS REFERENCE TO RELATED APPLICATIONS

This application is a nonprovisional application of and claims priority from U.S. provisional patent application Ser. No. 63/167,478 filed on Mar. 29, 2021. The foregoing application is incorporated in its entirety herein by reference.

### FIELD OF THE INVENTION

The invention relates to compositions for providing electrolytes. More particularly, the invention relates to electrolyte-containing beverage compositions for supplying a drinkable source of electrolytes and for replenishing electrolytes in a human or animal consuming the beverage composition and for providing other beneficial health effects related to digestion, nutrient absorption, pain prevention and alleviation, blood pressure reduction, and anxiety relief.

### BACKGROUND

Sports and energy drinks are often consumed by athletes, exercisers, and other persons to maintain or to replenish a healthful amount of electrolytes in the body, which are often lost before, during, or after physical exertion via perspiration and urination. The human body requires certain levels of electrolytes for maintenance of normal metabolic functions.

In some instances, drinks, shakes, and drink powders or other drink mixes are combined with other ingredients that provide additional health effects. However, current health drink offerings do not include any examples that provide or replenish electrolytes while also providing other beneficial health effects related to digestion, nutrient absorption, pain prevention and alleviation, blood pressure reduction, and anxiety relief.

A need exists for ingestible electrolyte-containing beverage compositions for supplying a drinkable source of electrolytes and for replenishing electrolytes in a human or animal consuming the beverage composition. A further need exists for ingestible beverage compositions containing ingredients for providing other beneficial health effects related to digestion, nutrient absorption, pain prevention and alleviation, blood pressure reduction, and anxiety relief. Still a further need exists for non-prescription, over-the-counter, ingestible electrolyte-containing beverage compositions for supplying a drinkable source of electrolytes and for replenishing electrolytes in a human or animal consuming the beverage composition, which also provides other beneficial health effects related to digestion, nutrient absorption, pain prevention and alleviation, blood pressure reduction, and anxiety relief.

### SUMMARY

Sports, energy, dietary, nutritional, and health beverages, drinks, and shakes and drink powders and mixes are often consumed by humans to restore electrolytes to the body that have been lost or depleted during physical exertion such as exercise, work, and sports participation. The beverage compositions of the present invention provide electrolytes and other beneficial health effects related to digestion, nutrient absorption, pain prevention and alleviation, blood pressure reduction, and anxiety relief. The composition includes minerals, mastica, vitamin C, and a cannabinoid compound. In liquid forms of the composition, it may further include water or it may include brine containing minerals. The cannabinoid compound can be cannabidiol or another cannabinoid compound that is not psychoactive. Methods for improving digestion, nutrient absorption, pain prevention and alleviation, blood pressure reduction, and anxiety relief involve consumption of beverage compositions containing the foregoing ingredients.

The compositions and methods of the present invention provide advantages to humans and animals by providing ingestible electrolyte-containing beverage compositions for supplying a drinkable source of electrolytes and for replenishing electrolytes in the human or animal consuming the beverage composition. The electrolyte-containing beverage compositions and related methods for improving health provide further advantages by including ingredients that produce other beneficial health effects related to digestion, nutrient absorption, pain prevention and alleviation, blood pressure reduction, and anxiety relief. The electrolyte-containing beverage compositions and related methods for improving health provide still further advantages by being distributable as non-prescription, over-the-counter, ingestible electrolyte-containing beverage compositions for supplying a drinkable source of electrolytes and for replenishing electrolytes in a human or animal consuming the beverage composition, which also provides other beneficial health effects related to digestion, nutrient absorption, pain prevention and alleviation, blood pressure reduction, and anxiety relief.

Accordingly, the invention relates to an ingestible composition for providing electrolytes and other beneficial health effects. The composition includes minerals, mastica, vitamin C, and a cannabinoid compound. The composition is or can include a powder that is mixable with water or another beverage.

In another aspect, the invention can feature the cannabinoid compound being or including cannabidiol.

In another aspect, the invention can feature the minerals being or including calcium, phosphorus, manganese, zinc, vanadium, and silicon.

In another aspect, the invention can feature the composition including about 50 mg to about 200 mg of the minerals.

In another aspect, the invention can feature the composition including about 200 mg to about 500 mg of the minerals.

In another aspect, the invention can feature the composition including about 500 mg to about 700 mg of the minerals.

In another aspect, the invention can feature the composition including about 6 mg to about 15 mg of the cannabinoid compound.

In another aspect, the invention can feature the composition including about 15 mg to about 25 mg of the cannabinoid compound.

In another aspect, the invention can feature the composition including about 25 mg to about 40 mg of the cannabinoid compound.

In another aspect, the invention can feature the composition including about 700 mg to about 900 mg of vitamin C.

In another aspect, the invention can feature the composition including about 900 mg to about 1,100 mg of vitamin C.

In another aspect, the invention can feature the composition including about 1,100 mg to about 1,250 mg of vitamin C.

In another aspect, the invention can feature the composition including about 40 mg to about 200 mg of mastica.

In another aspect, the invention can feature the composition including about 200 mg to about 400 mg of mastica.

In another aspect, the invention can feature the composition including about 400 mg to about 600 mg of mastica.

The invention also relates to a liquid ingestible composition for providing electrolytes and other beneficial health effects. The composition includes brine water that includes minerals, mastica, vitamin C, and a cannabinoid compound.

In another aspect, the invention can feature further including a second liquid, wherein the second liquid is a beverage with which the other ingredients are mixed.

In another aspect, the invention can feature the cannabinoid compound being or including cannabidiol.

In another aspect, the invention can feature the minerals being or including calcium, phosphorus, manganese, zinc, vanadium, and silicon.

The invention also relates to a method for providing electrolytes and other beneficial health effects. The method includes the steps of: (a) providing an ingestible composition, in dry or liquid form, that includes minerals, mastica, vitamin C, and a cannabinoid compound; and (b) ingesting the ingestible composition in a recommended dosage at a recommended interval of time.

Unless otherwise defined, all technical terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents and other references mentioned herein are incorporated by reference in their entirety. In the case of conflict, the present specification, including definitions will control.

### DETAILED DESCRIPTION

The present invention is best understood by reference to the detailed drawings and description set forth herein. Embodiments of the invention are discussed below with reference to the drawings; however, those skilled in the art will readily appreciate that the detailed description given herein with respect to these figures is for explanatory purposes as the invention extends beyond these limited embodiments. For example, in light of the teachings of the present invention, those skilled in the art will recognize a multiplicity of alternate and suitable approaches, depending upon the needs of the particular application, to implement the functionality of any given detail described herein beyond the particular implementation choices in the following embodiments described and shown. That is, numerous modifications and variations of the invention may exist that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

The present invention should not be limited to the particular methodology, compounds, materials, manufacturing techniques, uses, and applications, described herein, as these may vary. The terminology used herein is used for the purpose of describing particular embodiments only, and is not intended to limit the scope of the present invention. As used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” may be a reference to one or more steps or means and may include sub-steps and subservient means.

All conjunctions used herein are to be understood in the most inclusive sense possible. Thus, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should be read as “and/or” unless expressly stated otherwise. Structures described herein are to be understood also to refer to functional equivalents of such structures. Language that may be construed to express approximation should be so understood unless the context clearly dictates otherwise.

Unless otherwise defined, all terms (including technical and scientific terms) are to be given their ordinary and customary meaning to a person of ordinary skill in the art, and are not to be limited to a special or customized meaning unless expressly so defined herein.

Terms and phrases used in this application, and variations thereof, especially in the appended claims, unless otherwise expressly stated, should be construed as open ended as opposed to limiting. As examples of the foregoing, the term “including” should be read to mean “including, without limitation,” “including but not limited to,” or the like; the term “having” should be interpreted as “having at least”; the term “includes” should be interpreted as “includes but is not limited to”; the term “example” is used to provide exemplary instances of the item in discussion, not an exhaustive or limiting list thereof; and use of terms like “preferably,” “preferred,” “desired,” “desirable,” or “exemplary” and words of similar meaning should not be understood as implying that certain features are critical, essential, or even important to the structure or function of the invention, but instead as merely intended to highlight alternative or additional features that may or may not be utilized in a particular embodiment of the invention.

Those skilled in the art will also understand that if a specific number of an introduced claim recitation is intended, such an intent will be explicitly recited in the claim, and in the absence of such recitation no such intent is present. For example, as an aid to understanding, the appended claims may contain usage of the introductory phrases “at least one” and “one or more” to introduce claim recitations; however, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C” is used, in general, such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.).

All numbers expressing dimensions, quantities of ingredients, reaction conditions, and so forth used in the specification are to be understood as being modified in all instances by the term “about” unless expressly stated otherwise. Accordingly, unless indicated to the contrary, the numerical parameters set forth herein are approximations that may vary depending upon the desired properties sought to be obtained.

The invention provides an ingestible electrolyte-containing beverage composition for supplying a drinkable source of electrolytes and for replenishing electrolytes in a human or animal consuming the beverage composition, which also provides other beneficial health effects related to digestion, nutrient absorption, pain prevention and alleviation, blood pressure reduction, and anxiety relief.

In exemplary embodiments, the ingestible composition includes water, minerals (or brine in liquid forms of the composition), mastica, vitamin C, and a cannabinoid compound. The cannabinoid compound can be or include cannabidiol or any other suitable non-psychoactive cannabinoid compound. The mastica (also known as mastic gum) is derived from resin or extracts of the plant Pistacia lentiscus. Mastic and the minerals or brine impart electrolytes to the beverage composition. Mastic also improves digestion and nutrient absorption.

In other embodiments, the ingestible composition may include minerals, mastica, and a cannabinoid compound. The composition is producible as a powder, which can be added to water to create a drink mix. In exemplary embodiments, the composition is produced in amounts of about 9.1 grams, which can be deposited into packets or other containers for storage before use. Doses in other amounts or quantities may also be produced and consumed. However, the amounts of each ingredient specified in the paragraphs below are based on an about 9.1 gram dose.

In liquid forms of the composition, the composition may further include water or it may include brine containing minerals. The other ingredients are mixed in the water or brine.

In liquid forms of the composition containing brine, the brine includes at least one mineral. The at least one mineral can be elemental or a compound such as a salt. The at least one mineral can be or can include ionic trace minerals. In an exemplary embodiment of the composition, the brine includes the following minerals: calcium, phosphorus, manganese, zinc, vanadium, and silicon. In other embodiments, the composition may include additional minerals. The at least one mineral can be an ionic trace mineral derived from salt or water obtained from the

Great Salt Lake in Utah or from other salt lakes located in other geographic locations. Brine water from the Great Salt Lake is used in some preferred embodiments of the composition due to its unique dissolved mineral content. In other preferred embodiments, the composition may include brine that is produced by the addition of minerals and compounds in amounts or quantities necessary to mimic the dissolved mineral content of the Great Salt Lake. In exemplary embodiments, the at least one mineral can be about 70 trace minerals.

In dry forms of the composition (e.g., powders and other mixes), the dry minerals include calcium, phosphorus, manganese, zinc, vanadium, and silicon. In other embodiments, the composition may include additional minerals. In exemplary embodiments, the composition includes about 50 to about 750 mg of minerals. For example, the composition may include about 39.9, 40, 40.1, 45, 46, 47, 48, 49, 49.5, 49.9, 50, 50.1, 50.5, 50.9, 60, 65, 70, 75, 80, 90, 100, 125, 150, 175, 200, 300, 350, 375, 380, 390, 391, 392, 393, 394, 395, 396, 397, 398, 399, 399.1, 399.5, 399.75, 399.9, 400, 400.1, 400.25, 400.5, 400.9, 401, 402, 403, 404, 405, 406, 407, 408, 409, 410, 420, 425, 450, 500, 600, 625, 650, 675, 680, 690, 700, 710, 720, 730, 739.9, 740, 749, 749.1, 749.2, 749.3, 749.4, 749.5, 749.6, 749.7, 749.8, 749.9, 750, 750.1, 750.5, 750.9, 751, 755, 760, 770, 780, 790, or 800 mg of minerals. In a most preferred embodiment, the composition can be include about 400 mg of minerals derived from brine from a natural water source.

In one embodiment, the composition may include about 50 to about 200 mg of minerals when fewer electrolytes are required or desired.

In another embodiment, the composition may include about 200 to about 500 mg of electrolytes, e.g., for consumption after exercise or strenuous work or other activity.

In still another embodiment, the composition may include about 500 to about 750 mg of minerals when a greater amount of electrolytes are required, e.g., for consumption when a person is dehydrated or after very strenuous exercise, work, or other activity.

In liquid forms of the composition, or when dry forms of the composition are added to water or another liquid, the brine becomes a liquid as the trace minerals are dissolved in the water or other liquid. The amount or quantity of mineral content in the brine can be similar or the same as described above for dry forms of the composition.

Cannabidiol (CBD) imparts numerous health benefits to the composition including properties related to pain prevention and alleviation, blood pressure reduction, and anxiety relief.

Other cannabinoid compounds may be included in the composition in addition to cannabidiol or in lieu thereof. In exemplary embodiments, the composition includes about 6 to about 40 mg of CBD. For example, the composition may include about 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 8, 9, 10, 11, 12, 15, 20, 25, 29, 30, 31, 32, 33, 34, 35, 36, 37, 38, 38.9, 39, 39.1, 39.2, 39.3, 39.4, 39.5, 39.6, 39.7, 39.8, 39.9, 40, 40.1, 40.2, 40.3, 40.4, 40.5, 40.6, 40.7, 40.8, 40.9, 50, or 50.1 mg of CBD or another cannabinoid. In a most preferred embodiment, the composition can be include about 20 mg of CBD.

In one embodiment, the composition may include about 6 to about 15 mg of CBD when the beneficial health effects of CBD are required or desired but for prevention and alleviation or reduction of only mild pain, mildly elevated blood pressure, and/or mild anxiety.

In another embodiment, the composition may include about 15 to about 25 mg of CBD, e.g., when the beneficial health effects of CBD are required or desired but for prevention and alleviation or reduction of moderate pain, moderate elevated blood pressure, and/or moderate anxiety.

In still another embodiment, the composition may include about 25 to about 40 mg of CBD when a greater amount of electrolytes are required, e.g., when the beneficial health effects of CBD are required or desired but for prevention and alleviation or reduction of more severe or persistent pain, higher or more persistent elevated blood pressure, and/or more severe or persistent anxiety.

Vitamin C provides beneficial health effects by supporting a strong immune system and providing energy. In exemplary embodiments, the composition includes about 700 to about 1,250 mg of vitamin C. For example, the composition may include about 599.9, 600, 610, 620, 630, 640, 650, 660, 670, 680, 690, 691, 692, 693, 694, 695, 696, 697, 698, 699, 699.1, 699.5, 699.9, 700, 700.1, 700.5, 700.9, 701, 705, 710, 720, 730, 740, 750, 760, 770, 780, 790, 800, 900, 1,000, 1,100, 1,199, 1,200, 1,210, 1,220, 1,230, 1,240, 1,241, 1,245, 1,246, 1,247, 1,248, 1,249, 1,249.1, 1,249.2, 1,249.3, 1,249.4, 1,249.5, 1,249.6, 1,249.7, 1,249.8, 1,249.9, 1,250, 1,250.1, 1,250.2, 1,250.3, 1,250.4, 1,250.5, 1,250.6, 1,250.7, 1,250.8, 1,250.9, 1,251, 1,255, 1,260, 1,270, 1,280, 1,290, or 1,300 mg of vitamin C. In one embodiment, the composition can be include about 700 mg of vitamin C.

In one embodiment, the composition may include about 700 to about 900 mg of vitamin C for general immune system support and for boosting energy.

In another embodiment, the composition may include about 900 to about 1,100 mg of vitamin C for immune system support when a person has minor cold symptoms and for boosting energy, e.g., for consumption after mild or moderate exercise, work, or other activity.

In still another embodiment, the composition may include about 1,100 to about 1,250 mg of vitamin C for immune system support when a person has severe cold or flu symptoms and for boosting energy, e.g., for consumption after very strenuous exercise, work, or other activity.

In exemplary embodiments, the composition includes about 40 to about 600 mg of mastica. For example, the composition may include about 39.9, 40, 40.1, 45, 46, 47, 48, 49, 49.5, 49.9, 50, 50.1, 50.5, 50.9, 60, 65, 70, 75, 80, 90, 100, 125, 150, 175, 200, 300, 350, 375, 380, 390, 391, 392, 393, 394, 395, 396, 397, 398, 399, 399.1, 399.5, 399.75, 399.9, 400, 400.1, 400.25, 400.5, 400.9, 401, 402, 403, 404, 405, 406, 407, 408, 409, 410, 420, 425, 450, 500, 510, 525, 550, 575, 580, 590, 595, 599, 599.1, 599.2, 599.3, 599.4, 599.5, 599.6, 599.7, 599.8, 599.9, 600, 600.1, 600.2, 600.3, 600.4, 600.5, 600.6, 600.7, 600.8, 600.9, 601, 602, 603, 604, 605, 606, 607, 608, 609, 610, 625, 650, 675, 690, or 700 mg of trace minerals. In a most preferred embodiment, the composition can be include about 40 mg of mastica.

In one embodiment, the composition may include about 40 to about 200 mg of mastica for general support of digestion and nutrient absorption.

In another embodiment, the composition may include about 200 to about 400 mg of mastica for improved digestion and nutrient absorption, e.g., after mild or moderate exercise, work, or other physical activity or after mild or moderate illness.

In still another embodiment, the composition may include about 400 to about 600 mg of mastica for improved digestion and nutrient absorption, e.g., after strenuous exercise, work, or other physical activity or after more severe illness.

In exemplary embodiments, the composition further includes a flavoring such as, for example, orange. The flavoring can be natural, artificial, or a combination of both.

The ingredients described above work in combination to produce a synergistic effect that improves several aspects of health of a human or animal including health benefits related to digestion and nutrient absorption as well as other beneficial healthful effects related to pain prevention and alleviation, blood pressure reduction, and anxiety relief.

In one embodiment, the ingestible composition includes 400 mg of minerals, 40 mg of mastica, 600 mg of vitamin C, and 20 mg of cannabidiol. This embodiment may also include flavoring. This embodiment may be produced in dry form such as a powder, granules, or tablets that are introduced into and mixed with water or another liquid beverage.

In another embodiment, the ingestible composition includes 400 mg of minerals, 40 mg of mastica, 1,000 mg of vitamin C, and 25 mg of cannabidiol. This embodiment may also include flavoring. This embodiment may be produced in dry form such as a powder, granules, or tablets that are introduced into and mixed with water or another liquid beverage.

The composition is produced as a drink or beverage in exemplary embodiments. In other embodiments, the composition can be produced as a shake, a drink powder or mix for addition to water or another liquid beverage, or in solid form as a dietary or nutritional supplement.

The invention also relates to a method for maintaining or replenishing electrolytes in the body of a human or an animal and for providing other healthful benefits and effects as described herein above. The method includes the step of consuming a composition containing the ingredients described elsewhere herein. The composition may be provided in a dry powder, e.g., a drink mix, that is mixed in water or another beverage and consumed, or the composition may be provided in liquid form, e.g., as a ready-to-drink beverage. The dosage may be 9.1 g or some other amount that is lesser or greater. The composition may be consumed at intervals such as once, twice, or three times daily, every other day, a predetermined number of days each week, weekly, biweekly, or after each instance of illness or strenuous activity.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An ingestible composition for providing electrolytes and other beneficial health effects, the composition comprising:
minerals;
mastica;
vitamin C; and
a cannabinoid compound;
wherein the composition comprises a powder that is mixable with water or another beverage.

2. The composition of claim 1, wherein the cannabinoid compound comprises cannabidiol.

3. The composition of claim 1, wherein the minerals comprises calcium, phosphorus, manganese, zinc, vanadium, and silicon.

4. The composition of claim 1, wherein the composition comprises about 50 mg to about 200 mg of the minerals.

5. The composition of claim 1, wherein the composition comprises about 200 mg to about 500 mg of the minerals.

6. The composition of claim 1, wherein the composition comprises about 500 mg to about 700 mg of the minerals.

7. The composition of claim 1, wherein the composition comprises about 6 mg to about 15 mg of the cannabinoid compound.

8. The composition of claim 1, wherein the composition comprises about 15 mg to about 25 mg of the cannabinoid compound.

9. The composition of claim 1, wherein the composition comprises about 25 mg to about 40 mg of the cannabinoid compound.

10. The composition of claim 1, wherein the composition comprises about 700 mg to about 900 mg of vitamin C.

11. The composition of claim 1, wherein the composition comprises about 900 mg to about 1,100 mg of vitamin C.

12. The composition of claim 1, wherein the composition comprises about 1,100 mg to about 1,250 mg of vitamin C.

13. The composition of claim 1, wherein the composition comprises about 40 mg to about 200 mg of mastica.

14. The composition of claim 1, wherein the composition comprises about 200 mg to about 400 mg of mastica.

15. The composition of claim 1, wherein the composition comprises about 400 mg to about 600 mg of mastica.

16. A liquid ingestible composition for providing electrolytes and other beneficial health effects, the composition comprising:
brine water comprising minerals;
mastica;
vitamin C; and
a cannabinoid compound.

17. The composition of claim 17, further comprising a second liquid, wherein the second liquid is a beverage with which the other ingredients are mixed.

18. The composition of claim 17, wherein the cannabinoid compound comprises cannabidiol.

19. The composition of claim 17, wherein the minerals comprises calcium, phosphorus, manganese, zinc, vanadium, and silicon.

20. A method for providing electrolytes and other beneficial health effects, the method comprising the steps of:
(a) providing an ingestible composition, in dry or liquid form, comprising:
minerals;
mastica;
vitamin C; and
a cannabinoid compound; and
(b) ingesting the ingestible composition in a recommended dosage at a recommended interval of time.
